(19) United States
(12) Patent Application Publication
(10) Pub. No.: US 2003/0040018 A1
(43) Pub. Date: Feb. 27, 2003

(54) Method for determining significant losses in bone density

(76) Inventor: Norman Bitterlich, Chemnitz (DE)

Correspondence Address: JORDAN AND HAMBURG LLP, 122 EAST 42ND STREET, SUITE 4000, NEW YORK, NY, 10168, US

(21) Appl. No.: 10/009,125

(22) PCT Filed: Apr. 17, 2001
(86) PCT No.: PCT/EP01/04335

(30) Foreign Application Priority Data
Apr. 28, 2000 (DE) 100 20 880.0

(51) Int. Cl. G01N 33/53; G01N 33/567; G06F 19/00; G01N 33/48; G01N 33/50

(52) U.S. Cl. 435/7.2; 702/19

(57) ABSTRACT

The aim of the invention is to provide a method for determining significant losses in bone density which is less cost-intensive, does not expose the patient to radiation and whose response time is shortened in terms of the interaction between osteoclasis and osteosteosis. According to the invention, this is achieved by using measuring values of real or mathematically simulated processes of bone density losses contained in electronic storage media, reflecting the temporal dependency of laboratory parameters with respect to practically or theoretically known clinical symptoms, as reference values during the process. Measuring values of bone markers are determined, using widespread laboratory techniques, from serum or urine samples during steps in which the samples are prepared such as the addition of antibodies, incubation stops, separation methods and insertion in analysis techniques. Said values are associated with losses in bone density and are written into an electronic data memory with the aid of an input marker. According to the invention, said values are used to determined significant losses in bone density.

The invention relates to a method for determining significant bone density losses.

In the interaction of bone destruction processes and bone formation processes, there may, on the average, be loss of bone substance and structure. In order to determine this loss and identify significant deviations from age-specific and sex-specific reference values, bone density measurements (osteodensitometry) are generally carried out. Using the methods of photon adsorption or computer tomography, the bone mineral density is measured at the radius, at the thigh bone (femur) or at the lumbar vertebral column (Vertebra lumbalis). Under the assumption of an exponential development of the bone density decrease, the loss can be estimated from bone density values measured at least at three different times. Intra-individual measurement fluctuations of up to 5 percent may be obtained. In order to keep these factors, which interfere with the determination of the results, as small as possible, sufficiently large intervals between the times of measuring the bone density should be selected, so that changes in the bone density can be detected adequately. At the earliest, therefore, information is available after one year.

Osteodensitometric methods of identifying bone density losses are expensive, cost intensive and expose the patient to radiation. Moreover, osteodensitometry is not transferable between different instruments and measurement methods are not standardized. At the present time, there are no recognized alternative methods with equivalent informative capability.

In the literature (P. Garnero and P. D. Dalmas, “Biochemical Markers of Bone Turnover” 1988, Endocrinology and Metabolism Clinics of North America, vol. 27, No. 2, pp. 303-322), laboratory parameters, which are particularly associated with bone density losses (bone markers), are investigated extensively with respect to their suitability for describing bone density loss quantitatively. Such parameters from the cell portion (osteoblasts), from the organic matrix of the bone (collagen, noncollageous proteins) or from the inorganic constituents of the bone admittedly can give an indication of bone loss. Until now, however, the information, provided by them, is not equivalent to the information provided by osteodensitometry. With that, the advantage of simplicity of this method of determining parameters in the serum or the urine cannot be utilized.

It is an object of the invention to develop a method for determining significant bone density losses, which is less cost intensive, does not expose the patient to radiation and shortens the period for interpreting the interaction of bone destruction processes and bone formation processes.

Pursuant to the invention, this objective is accomplished owing to the fact that measurement values of real or mathematically simulated bone density loss processes, which are present in electronic storage media and, as a function of time, reflect laboratory parameters of practically or theoretically known clinical signs and symptoms, are used as reference values for the process, that bone marker values of serum or urine samples, associated with bone density losses and measured by common laboratory techniques, are determined by sample preparation steps such as

treating with antibodies

incubation steps

separation procedures

using analytical techniques

and recorded over an input mask in an electronic data memory, are used for determining significant bone density losses,

a) at the time of the analysis, all N available patient-related data being copied from the data memory over an interrogation function and made available for the further processing (measured values M (tₙ; k) of the K in the laboratory of the bone marker, determined after step x of the process at times t₁ . . . tₙ)

b) the measured values of the bone markers with respect to the first line in the Table being normalized according to the equation

$$\begin{matrix}{M}^{*} ( {t}_{n} ; k ) = \frac{M ( {t}_{n} ; k ) - M ( {t}_{1} ; k )}{M ( {t}_{1} ; k )} & k = 1 , … , K\end{matrix} ; n = 1 , … , N$$

and the measurements as a function of time being converted into months,

c) the normalized measured value being converted into a scalar quantity D(tₙ) for the graduated description of the course of the bone density, the equation

$$D ( {t}_{n} ) = \sqrt{{\sum }_{k = 1}^{K} W k \cdot {( {M}^{*} ( {t}_{n} ; k ) )}^{2}}$$

being used as functions for the graduated description of the course of the relationship

d) from the evaluations of the progress determined, evaluations of the progress for those time sections of

$${D}^{*} ( t ) = \frac{( {t}_{n} - t ) \cdot D ( n - 1 ) + ( t - {t}_{n - 1} ) \cdot D ( n )}{{t}_{n} - {t}_{n - 1}} , {t}_{ε} [ {t}_{n - 1} , {t}_{n} ]$$

being calculated by interpolation, for which reference values are available,

e) from the interpolated evaluations of the progress, similarity dimensions being calculated, the function

$${A}_{j} ( t ) = {\sum }_{m = 1}^{M} \frac{{t}_{m}}{{t}_{M}} \cdot {V}_{m} \cdot ( {( {R}_{j} ( {t}_{m} - {D}^{*} ( {t}_{m} ) ) )}^{2}$$

being used to calculate a similarity dimension between the data, which is to be investigated, and all the reference values, available in the database and, at the same time, similarity dimensions to the reference values and to the time in months being found;

f) from the similarity dimensions for all reference values, those reference values being determined, which have a high similarity in the mathematical sense, such as the

greatest similarity:

$$greatest similarity : {A}^{*} = {min}_{j > 1 , … I} \{ {A}_{j} \}$$

positive alternative

$$positive alternative ( + ) {A}^{+} = {min}_{j - 1 , … , I , Aj \neq {A}^{*} , R j ( t N ) > D ( t N )} \{ {A}_{j} \}$$

negative alternative

$$negative alternative ( - ) {A}^{-} = {min}_{j = 1 , … , J , Aj \neq {A}^{*} , R j ( t N ) < D ( t N )} \{ {A}_{j} \}$$

with subsequent output of the type description as text component for describing the situation;

g) the prediction being derived from these three reference values, if B₁=A*, B₂=A⁺, B₃=A⁻, the following expression

$$R ( t ) = \frac{1}{{\sum }_{i = 1}^{3} {B}_{i}} \cdot {\sum }_{j = 1}^{3} ( ( {\sum }_{i = 1}^{3} {B}_{i} - {B}_{j} ) \cdot {R}_{j} ( t ) )$$

being used for the predicted value at time t

h) the degrees of freedom for the specification of the model, given as functional parameters in the functional relation of D(tₙ) and Aⱼ(t) being occupied by standard specifications and fitted by statistical analysis of the reference values to practical experience for optimizing the quantitative prediction of the bone density loss;

i) the time being calculated, at which, according to this prediction strategy, the percentage deviation is greater than a specified threshold value, this time being the starting point for planning the scheduling of the next investigation.

Advantageously, the degrees of freedom, given as function parameters in the functional relationship of D(tₙ) and Aⱼ(t), are filled in by the mathematical method of least squares so that specified sequences are taken into consideration in the best way for reference values.

The reference values used can be values from an assumed analytical mathematical course (exponential function), from empirically based values from imaginary, assumed processes and from concrete, measured values from patients with known situations.

In the following, the invention is explained in greater detail by means of an example.

Osteocalcin, parathyroid hormone and alkaline phosphatase are used as bone markers. Data for the bone markers are obtained from serum or urine samples using well-known laboratory techniques (HPLC, RIA, ELISA). For this purpose, sample preparation steps such as

treatment with antibodies

incubation steps

separation methods

use in analytical techniques

are necessary in order to obtain, after the measurement, a quantitative value in the parameter-specific unit as a monitor or printer output or as an electronically available numerical value. These laboratory values obtained are stored in an electronic database over an input mask. It is a prerequisite of the method that reference values are known. Reference values can be values calculated theoretically from an analytically mathematically assumed course (exponential function) or empirically based values from imaginary, assumed processes or concrete values from patients with known situations. These reference values are available for specified times and can be used in the analysis only within the scope of the time horizon determined therewith. In the sample, the exponential function R(t)=a(1−e^{-bt}) is used to describe bone density losses. The parameters a and b then describe the speed and the degree of loss (t in month). Some reference examples are listed in the following Table:

| Months | | | | | Type | | Type |
|---|---|---|---|---|---|---|---|
| After | | Type I | Type II | Type | IV | Type V | VI |
| First | | a = 1 | a = 0.5 | III | a = 2 | a = 1 | a = 2 |
| Measure- | Type 0 | b = | b = | a = 0.5 | b = | b = | b = |
| ment | a = 0 | 0.01 | 0.05 | b = 0.1 | 0.01 | 0.05 | 0.05 |
| 0 | 0.00 | 0.00 | 0.00 | 0.00 | 0.00 | 0.00 | 0.00 |
| 6 | 0.00 | 0.06 | 0.13 | 0.23 | 0.12 | 0.26 | 0.52 |
| 12 | 0.00 | 0.11 | 0.23 | 0.35 | 0.23 | 0.45 | 0.90 |
| 18 | 0.00 | 0.16 | 0.30 | 0.42 | 0.33 | 0.59 | 1.19 |
| 24 | 0.00 | 0.21 | 0.35 | 0.45 | 0.43 | 0.70 | 1.40 |
| 30 | 0.00 | 0.26 | 0.39 | 0.48 | 0.52 | 0.78 | 1.55 |
| 36 | 0.00 | 0.30 | 0.42 | 0.49 | 0.60 | 0.83 | 1.67 |
| 42 | 0.00 | 0.34 | 0.44 | 0.49 | 0.69 | 0.88 | 1.76 |
| 48 | 0.00 | 0.38 | 0.45 | 0.50 | 0.76 | 0.91 | 1.82 |
| 54 | 0.00 | 0.42 | 0.47 | 0.50 | 0.83 | 0.93 | 1.87 |
| 60 | 0.00 | 0.45 | 0.48 | 0.50 | 0.90 | 0.95 | 1.90 |

This is followed by the steps below

a) the values measured and those obtained on three prior occasions are given in the Table below:

| | | Osteocalcine in | PTH | AP |
|---|---|---|---|---|
| No. | Date | μg/L | in ng/L | in U/L |
| 1 | May 30, 1996 | 9.8 | 24.8 | 90 |
| 2 | Jan. 29, 1997 | 10.9 | 34.6 | 86 |
| 3 | Feb. 16, 1998 | 12.6 | 32.0 | 104 |
| 4 | Mar. 2, 1999 | 12.4 | 34.0 | 107 |

measured values M(tₙ;k) for n=1, . . . , 4 and k=1, . . . , 3

b) the values obtained by measurement are normalized with respect to the first line in the Table according to the following equation

$${M}^{*} ( {t}_{n} ; k ) = \frac{M ( {t}_{n} ; k ) - M ( {t}_{i} ; k )}{M ( {t}_{i} ; k )}$$

and the time interval between the measurements is converted into months;

| No. | Month | M*(t;1) | M*(t;2) | M*(t;3) |
|---|---|---|---|---|
| 1 | 0.0 | 0.00 | 0.00 | 0.00 |
| 2 | 8.0 | 0.11 | 0.40 | −0.04 |
| 3 | 20.6 | 0.29 | 0.29 | 0.16 |
| 4 | 33.1 | 0.27 | 0.37 | 0.19 |

Normalized values measured M(tₙ;k) for n=1, . . . ,4 and k=1 . . . , 3

c) the normalized measured value is converted into a scalar quantity for the graduated description of the bone density loss, the equation

$$D ( {t}_{n} ) = \sqrt{{\sum }_{k = 1}^{K} W k \cdot {( {M}^{*} ( {t}_{n} ; k ) )}^{2}}$$

being used (K=3; n=1, . . . ,3) as a function of the graduated description of the progress. Under the standard setup, it is, of course assumed that w=1 for all weighting factors;

Evaluations of Progress at Time t

d) From the evaluations of the progress obtained, evaluations of the progress for those time intervals of

$${D}^{*} ( t ) = \frac{( {t}_{n} - t ) \cdot D ( n - 1 ) + ( t - {t}_{n - 1} ) \cdot D ( n )}{{t}_{n} - {t}_{n - 1}} , {t}_{∈} [ {t}_{n - 1} , {t}_{n} ]$$

are calculated by interpolation, for which reference values are available;

Interpolated Evaluations of Progress at Imaginary Measurement Times at 6-Month Intervals.

e) from the interpolated evaluations of the progress, similarity dimensions are calculated, the function

$${A}_{j} ( t ) = {\sum }_{m = 1}^{M} \frac{{t}_{m}}{{t}_{M}} \cdot {V}_{m} \cdot ( {( {R}_{j} ( {t}_{m} - {D}^{*} ( {t}_{m} ) ) )}^{2} , j = 1 , … , 6 ; M = 6 ,$$

being used for calculating a similarity dimension between the data, which is to be investigated, and all reference values available in the database, the following similarity dimensions being found. Under a standard setup, V=1 for all weighting factors.

| | | Type | Type | Type | Type | Type | Type | Type |
|---|---|---|---|---|---|---|---|---|
| Month | | 0 | I | II | III | IV | V | VI |
| 0 | | | | | | | | |
| 6 | 0.32 | 0.10 | 0.07 | 0.04 | 0.01 | 0.04 | 0.00 | 0.04 |
| 12 | 0.43 | 0.24 | 0.14 | 0.06 | 0.01 | 0.06 | 0.00 | 0.24 |
| 18 | 0.44 | 0.36 | 0.17 | 0.06 | 0.01 | 0.05 | 0.02 | 0.72 |
| 24 | 0.47 | 0.48 | 0.19 | 0.06 | 0.01 | 0.04 | 0.07 | 1.41 |
| 30 | 0.49 | 0.63 | 0.21 | 0.06 | 0.00 | 0.03 | 0.14 | 2.25 |

Similarity Dimensions for all Reference Values and all Times up to 30 Months

The instructions for the calculations explicitly do not represent a function for estimating the parameters a and b of the exponential function, since general reference values do not have to satisfy these instructions;

f) from the similarity dimensions for all reference values, those reference values are determined, which have a high similarity in the mathematical sense, such as the

$$\begin{matrix}greatest similarity : & {A}^{*} = {min}_{j - I , … J} \{ {A}_{j} \} & = 0 , 00 \\ positive alternative ( + ) & {A}^{+} = {min}_{j \cdot I , … J , Aj \neq {A}^{*} \cdot Rj ( iN ) > D ( iN )} \{ {A}_{j} \} & = 0 , 03 \\ negative alternative ( - ) & {A}^{-} = {min}_{j \cdot I , … J , Aj \neq {A}^{*} \cdot Rj ( iN ) < D ( iN )} \{ Aj \} & = 0 , 06\end{matrix}$$

In accordance with these selection instructions, Type III is recognized as the reference value and Type IV and Type II are selected as positive and negative, alternative reference value respectively;

g) The prediction is derived from these three reference value changes. If, in order to simplify the notation, B₁=A*, B₂=A⁺, B₃=A⁻, the following is used as the predicted value at time t

$$R ( t ) = \frac{1.}{{\sum }_{i = 1}^{3} {B}_{i}} \cdot {\sum }_{j = 1}^{3} ( ( {\sum }_{i = 1}^{3} {B}_{i} - {B}_{j} ) \cdot {R}_{j} ( t ) )$$

h) The degrees of freedom for the specification of the model, given as functional parameters in the functional relation of D(tₙ) and Aⱼ(t), are filled in by reference values, in order to achieve a quantitative prediction of the bond density loss;

| | | Prediction | Prediction | | Deviation |
|---|---|---|---|---|---|
| | Prediction | for | for | | from |
| | for | Alternatives | Alternatives | Predicted | Prediction |
| Month | Type III | (+) | (−) | Value R(t) | in % |
| 36 | 0.49 | 0.60 | 0.42 | 0.52 | 6.1 |
| 42 | 0.49 | 0.69 | 0.44 | 0.55 | 12.2 |
| 48 | 0.50 | 0.76 | 0.45 | 0.58 | 16.0 |
| 54 | 0.50 | 0.83 | 0.47 | 0.61 | 22.0 |
| 60 | 0.50 | 0.90 | 0.48 | 0.63 | 26.0 |

Predicted Value

The certainty of this prediction is characterized by the percentage deviation of the average predicted value R(t), using only the suitable reference value, ascertained with the similarity dimension factors A₁, in the example, prediction for

Type III, which has the greatest similarity A*

i) The time is calculated, at which, according to this prediction strategy, the percentage deviation is greater than a specified threshold value, this time being the starting point for planning the scheduling for the subsequent investigation. For this purpose, this value is put out over a monitor or printer and transmitted by remote data transmission to the treating physician. In the case of the present example, the threshold value is assumed with a 10% deviation. Forty months after the first measurement, a deviation greater than 10% is then found for the first time. The time for the next measurement should therefore not be later than 7 months after the values for the bone markers are determined.

| | | Prediction | Prediction | | Deviation |
|---|---|---|---|---|---|
| | Prediction | for | for | | from |
| | for | Alternatives | Alternatives | Predicted | Prediction |
| Month | Type III | (+) | (−) | Value R(t) | in % |
| 40 | 0.49 | 0.66 | 0.43 | 0.54 | 10.0 |

Deviation of the predicted value greater than 10%.

1. A method for determining significant bone density losses, wherein measurement values of real or mathematically simulated bone density loss processes, which are present in electronic storage media and, as a function of time, reflect laboratory parameters of practically or theoretically known clinical signs and symptoms, are used as reference values for the process, and bone marker values of serum or urine samples, associated with bone density losses and measured by common laboratory techniques, are determined by sample preparation steps such as
treating with antibodies
incubation steps
separation procedures
using analytical techniques
and recorded over an input mask in an electronic data memory, are used for determining significant bone density losses,
a) at the time of the analysis, all N available patient-related data being copied from the data memory over an interrogation function and made available for the further processing (measured values M (tₙ; k) of the K in the laboratory of the bone marker, determined after step x of the process at times t₁ . . . tₙ)
b) the measured values of the bone markers with respect to the first line in the Table being normalized according to the equation
$${M}^{*} ( {t}_{n} ; k ) = \frac{M ( {t}_{n} ; k ) - M ( {t}_{1} ; k )}{M ( {t}_{1} ; k )} k = 1 , … , K ; n = 1 , … , N$$
and the measurements as a function of time being converted into months,
c) the normalized measured value being converted into a scalar quantity D(tₙ) for the graduated description of the course of the bone density, the equation
$$D ( {t}_{n} ) = \sqrt{{\sum }_{k = 1}^{K} Wk \cdot {( {M}^{*} ( {t}_{n} ; k ) )}^{2}}$$
being used for the graduated description of the course of the relationship
d) from the evaluations of the progress determined, evaluations of the progress for those time sections of
$${D}^{*} ( t ) = \frac{( {t}_{n} - t ) \cdot D ( n - 1 ) + ( t - {t}_{n - 1} ) \cdot D ( n )}{{t}_{n} - {t}_{n - 1}} , {t}_{∈} [ {t}_{n - 1 ,} {t}_{n} ]$$
being calculated by interpolation, for which reference values are available,
e) from the interpolated evaluations of the progress, similarity dimensions being calculated, the function
$${A}_{j} ( t ) = {\sum }_{m = 1}^{M} \frac{{t}_{m}}{{t}_{M}} \cdot {V}_{m} \cdot ( {( {R}_{j} ( {t}_{m} - {D}^{*} ( {t}_{m} ) ) )}^{2}$$
being used to calculate a similarity dimension between the data, which is to be investigated, and all the reference values, available in the database and, at the same time, similarity dimensions to the reference values and to the time in months being found,
f) from the similarity dimensions for all reference values, those reference values being determined, which have a high similarity in the mathematical sense, such as the
greatest similarity:
$$greatest similarity : {A}^{*} = {min}_{j + 1 , … , \int } \{ {A}_{j} \}$$
$$positive alternative ( + ) {A}^{+} = {min}_{j - 1 , … , \int , Λ j + Λ * , Rj ( t N ) > D ( t N )} \{ {A}_{j} \}$$
$$negative alternative ( - ) {A}^{-} = {min}_{j - 1 , … , \int , Λ j + Λ * , Rj ( t N ) < D ( t N )} \{ {A}_{j} \}$$
with subsequent output of the type description as text component for describing the situation;
g) the prediction being derived from these three reference values, if B₁=A*, B₂=A⁺, B₃=A⁻,
the following expression
$$R ( t ) = \frac{1}{{\sum }_{j = 1}^{J} {B}_{i}} \cdot {\sum }_{j = 1}^{J} ( ( {\sum }_{j = 1}^{J} {B}_{i} - {B}_{j} ) \cdot {R}_{j} ( t ) )$$
being used for the predicted value at time t
h) the degrees of freedom for the specification of the model, given as functional parameters in the functional relation of D(tₙ) and Aⱼ(t) being occupied by standard specifications and fitted by statistical analysis of the reference values to practical experience for optimizing the quantitative prediction of the bone density loss;
i) the time being calculated, at which, according to this prediction strategy, the percentage deviation is greater than a specified threshold value, this time being the starting point for planning the scheduling of the next investigation.

2. The method of claim 1, wherein the degrees of freedom, given as function parameters in the functional relationship of D(tₙ) and Aⱼ(t), are filled in by the mathematical method of least squares so that specified sequences are taken into consideration in the best way for reference values.

3. The method of claim 1, wherein reference values are used, which are calculated values from an analytical mathematically assumed course (exponential functions).

4. The method of claim 1, wherein reference values are used, which are empirical values from imaginary, assumed processes.

5. The method of claim 1, wherein reference values are used, which are concrete values from patients with known situations.
